(19) United States
(12) Patent Application Publication
(10) Pub. No.: US 2025/0228792 A1
(43) Pub. Date: Jul. 17, 2025

(54) WATER-BASED FORMULATION OF DISCREET LIQUID PATCH FOR THE PAIN-FREE TREATMENT OF COLD SORES

(71) Applicant: Adam Murday, Midland (CA)

(72) Inventor: Adam Murday, Midland (CA)

(21) Appl. No.: 18/628,763

(22) Filed: Apr. 7, 2024

Related U.S. Application Data
(60) Provisional application No. 63/459,953, filed on Apr. 17, 2023.

(51) Int. Cl. A61K 9/70; A61K 9/00; A61K 31/045; A61K 31/14; A61K 31/198; A61K 31/522; A61K 33/06; A61K 33/30; A61K 45/06; A61K 47/02; A61K 47/10; A61K 47/14; A61K 47/18; A61K 47/22; A61K 47/32; A61K 47/34; A61K 47/38

(52) U.S. Cl. CPC A61K 9/7015; A61K 31/045; A61K 31/14; A61K 31/198; A61K 31/522; A61K 33/06; A61K 33/30; A61K 45/06; A61K 47/02; A61K 47/10; A61K 47/14; A61K 47/183; A61K 47/22; A61K 47/32; A61K 47/34; A61K 47/38; A61K 9/0014

(57) ABSTRACT

This application discloses a water-based formulation for a discreet liquid patch offering pain-free cold sore treatment. The patch adheres to the lesion without hydrocolloid particles or backing layers. It includes an adhesive layer with a water-based polymer and an active ingredient such as Docosanol, Acyclovir, Penciclovir, or Lysine. The adhesive layer, 5 to 1,300 microns thick, uses biocompatible polymers (e.g., acrylate monomers, polyethylene oxide, or polyvinyl alcohol) to ensure skin compatibility and minimize allergic reactions. The water-based adhesive provides non-irritating adhesion while delivering active ingredients. This formulation offers a convenient, sting-free treatment option, applicable until complete re-epithelialization.

### BACKGROUND

### Field of the Invention

The present invention provides a water-based formulation of a discreet liquid patch that offers a pain-free and effective treatment for cold sores. The patch is designed to be applied to the affected lesion and remain in contact with it for a time sufficient for complete re-epithelialization of the sore. The patch includes an adhesive layer that is substantially free of hydrocolloid particles and does not have any backing layer. The formula comprises an active compound or ingredients from the group consisting of Docosanol, Acyclovir, Penciclovir, Lysine, or similar, and a water-based adhesive.

The water-based adhesive used in the formulation may be any suitable water-soluble polymer that exhibits excellent adhesion properties, allowing the patch to adhere to the skin and remain in place for the desired duration of treatment. The adhesive may include polymers such as polyvinyl alcohol, polyethylene oxide, polyacrylamide, polyvinylpyrrolidone, or other water-based polymers. These water-based adhesives provide a non-irritating, comfortable, and safe adhesion to the skin while delivering the active ingredients.

The thickness of the patch may range from 5 microns to 1,300 microns. The invention uses biocompatible polymer materials in the adhesive layer, such as acrylate monomers polymerized through a micro-emulsion polymerization process, to ensure compatibility with the skin and reduce the risk of skin irritation or allergic reactions. However, other suitable biocompatible polymers may also be used, such as polyethylene oxide, polyvinylpyrrolidone, or polyvinyl alcohol, or any other suitable biocompatible polymer that meets the desired properties of the patch.

This formulation is specifically designed for the treatment of cold sore lesions, providing a unique and novel approach to managing cold sore outbreaks. The water-based adhesive in combination with active ingredients offers a non-invasive, comfortable, and convenient treatment option for cold sores, with the distinct benefit over existing prior art of being sting-free.

### DESCRIPTION OF THE RELATED ART

Herpes Labialis, commonly known as cold sores, is a type of infection caused by the herpes simplex virus (HSV) that primarily affects the lips. HSV-1 and 2 infections are characterized by a high frequency of recurrence and are highly contagious. Cold sores can cause physical pain, discomfort, and disfigurement, especially in those patients with a high frequency of recurrence. Those afflicted with the virus often feel embarrassed by the appearance of the lesions.

The global seroprevalence of HSV-1 in adults is currently estimated to be between 67% and 87%, resulting in more than 3.7 billion cases of cold sores annually. The Centers for Disease Control and Prevention (CDC) estimates that the prevalence of HSV-1 in the United States for people between the age of 14 and 49 years old was 47.8% between 2015-2016, with the highest prevalence in individuals aged 60 years and older [1]. Cold sores, which are caused by the herpes simplex virus, are a common manifestation of HSV-1 infection. The first sign of a cold sore is usually a tingling, burning, or itching sensation on or around the lips, beginning about 12-24 hours before the cold sore develops. The area becomes red, swollen, and painful as the blisters form. Over 2-3 days, the blisters rupture and ooze fluid that is clear or slightly yellow. After the blisters break, a scab forms that can last several days. Treatment options for cold sores include antiviral medication, which can be applied directly to the sores or taken orally [3]., and OTC topical applications such as creams, gels and material backed patches. Furthermore, the majority of people who have had cold sores will suffer recurring outbreaks, with more than 50 million adults in the United States experiencing two or more outbreaks per year. A typical outbreak will generally regress within 7-10 days, with complete healing in 12-14 days, although a scar or erythema may persist.

The uncomfortable and sensitive lesions of Herpes Labialis are painful when touched and interfere with patients' daily routines, such as eating and drinking. Additionally, the unsightly nature of the lesions, coupled with the discomfort, often causes patients to feel embarrassed.

Although 90% of the population is exposed to the virus in their lifetime, there is a social element of antipathy to the virus that adds to this embarrassment.

Patients often have trouble restraining themselves from touching cold sore lesions which can result in the spread of the infection, exacerbate the lesion, and increase healing times. However, creating a barrier or a layer on the surface of the lesion that protects against outside influences, such as bacteria, dirt, and physical agitation, can reduce the sensitivity to the affected area and improve the patient's ability to perform daily routines. The most common method to create this layer on the surface of the lesion is by using a hydro-colloidal based patch. Patches create a preventative barrier and can eliminate/reduce this influence with less sensation experience.

The formulation or composition of the patch is an essential part of providing a lighter and more flexible/stable patch that is less prominent while still providing significant healing and usage time properties. Most commercial patches include a formulation consisting of an adhesive layer and a material backing substrate layer. The adhesive layer is typically made from a hydrocolloidal composition that combines with moisture produced by the lesion and turns into a gel-like substance, while the backing layer provides structural support. Some commercial patches are also non-hydrocolloid compositions; however, they are non-medicated. Abreva is the most well-known non-hydrocolloid-based patch and is non-medicated. Common hydrocolloidal patches include Compeed and Polysporin, both non-medicated.

Canadian Patent No. CA 2609259 discloses a hydrocolloid base patch, which includes a backing layer for treating Herpes Labialis. U.S. patent application No. 20070026056A1 also discloses a backing of a flexible sheet of water-insoluble material.

Hydrocolloid base patch formulations with or without active ingredients can become unsightly as the lesion underneath produces a buildup of organic material in a gel-like substance, causing the patch to become thicker and often destabilizing the adhesion of the patch and resulting in a more visible patch.

Non-hydrocolloidal base patch formulations without active ingredients would not experience the gelling of organic materials but they do not effectively manage the moisture produced by the lesion. The moisture builds up beneath the patch and destabilizes the adhesive, separating the patch from the skin. It becomes more visible and uncomfortable, and likewise a hotspot for activity and increased bacterial load. Additionally, the lack of active ingredients slows down the healing process. The application of hydrocolloid base and non-hydrocolloid base patches is not easy and can interfere with the physical appearance, potentially aggravating the area further, and increasing the risk of infection at the affected site.

U.S. Pat. No. 11,311,494B2, which focuses on alcohol-based compositions for a discreet liquid cold sore patch to treat cold sore lesions addresses the above-mentioned challenges with the existing product landscape. However, such formulations can be problematic because the stinging sensation of alcohol-based adhesives can be intolerable for patients, especially young children. Therefore, there is a need for a pain-free water-based compound/formulation that can effectively manage the moisture produced by the lesion, speed up the healing process, and reduce the risk of infection while maintaining a pain-free (sting-free) formulation.

### SUMMARY

The present invention provides a water-based translucent protective barrier that also delivers an anti-viral compound for the treatment of cold sores/viral lesions. A major benefit of water-based formulations is that the resulting product is sting-free while providing the additional benefits of being flexible, providing a lighter and more stable patch compared to the industry standard products, with significant healing and usage time properties without triggering more infection. The liquid discreet patch formulation includes a translucent liquid adhesive, at least one active ingredient, a non-active ingredient, and an optional anti-bacterial ingredient. The translucent liquid adhesive is composed of a water-based formulation, defined as being between 75-90% water by weight, which includes the non-active ingredients. The liquid discreet patch does not require an additional backing layer.

The formulation of the liquid discreet patch includes mixing one or more non-active ingredients at specific temperature and mixing conditions to form a translucent liquid adhesive. Other ingredients such as the active ingredients and the anti-bacterial compound can be mixed separately at specific temperature and mixing conditions to form a translucent liquid. Furthermore, the two separate mixtures combine to form the liquid discreet patch formulation.

A preferred embodiment of the invention comprises the active ingredient selected from a group consisting of anti-viral compounds, amino acids, and vitamins such as Docosanol, L-lysine, Acyclovir, Penciclovir, Zinc, and Magnesium. Whereas, at least one active ingredient can be combined with the water-based translucent liquid adhesive to form the translucent liquid discreet patch. The optional anti-bacterial compounds such as Benzalkonium Chloride and Benzethonium Chloride and/or natural compounds like Silver, Tea tree oil, Honey, Eucalyptus oil, Garlic, Cinnamon, Turmeric, Manuka oil, Aloe vera, Grapefruit seed extract can be mixed with the liquid discreet patch formulation.

According to another aspect of the invention, the liquid discreet patch is applied as a liquid on top of a part of the skin/anatomical lesion surface, principally the lips and/or facial area, for the treatment of viral lesions or cold sores. Once the patch is air-dried, it forms a protective translucent film barrier to prevent additional spread of the virus and contamination from bacteria, microorganisms and fine particles.

According to another aspect of the invention, the translucent nature of the liquid patch reduces the physical appearance of lesions, and the nature of the patch allows for the use of facial makeup without negatively affecting the properties of the patch. The patch is maintained in contact with the cold sore/lesion to improve healing time, reduce the spread of the virus, and reduce the time to complete re-epithelization, as compared to an untreated cold sore/viral outbreak.

A principal characteristic of the present invention, is its use of a water-based adhesive in order to provide a product that is pain free during application as well as during standard wear.

The preceding and additional characteristics of the invention are fully explained in the claims, with the following description detailing one or more examples of how the invention may be used. The description of the invention provided herein is only illustrative and not exhaustive, and it should be understood that the principles of the invention can be employed in various ways.

### DETAILED DESCRIPTION

The present invention concerns a pain-free, water-based composition formulated for the effective treatment of cold sores and viral lesions. The composition is specifically designed to offer a pain-free product that accelerates the healing process, minimizes discomfort, and prevents secondary spread of infection.

More specifically, the present invention relates to formulation of the liquid discreet patch comprising of a translucent liquid adhesive, at least one active ingredient, a non-active ingredient, and an optional anti-bacterial compound. The translucent liquid adhesive is composed of a water-base formulation, that is, where water is used as the primary solvent, and/or diluent, and includes the non-active ingredient(s), whereas, the liquid discreet patch does not require an additional backing layer.

According to a preferred embodiment, the formulation comprises, the translucent liquid adhesive, which is composed of a water-based adhesive, for the specific purpose of reducing and/or eliminating pain most often represented by a stinging sensation at the time and area of application. The water base composition of the translucent liquid adhesive further includes at least one non-active ingredient selected from the group consisting of polyurethane, cyanoacrylate, acrylates, vinyl acetate, silicone, 2-ethylhexyl acrylate (2-EHA), N-vinylpyrrolidone (NVP), acrylic acid, vinyl acetate, butyl acrylate, polyvinyl alcohol (PVA), polyvinylpyrrolidone (PVP), polyacrylamide (PAM), hydroxyethyl cellulose (HEC), carboxymethyl cellulose (CMC), ethylene-vinyl acetate (EVA), acrylonitrile-butadiene-styrene (ABS), styrene-acrylic (SA), vinyl acetate-ethylene (VAE), and acrylic acid-ethylene (AE). More specifically, composition comprises suitable weights of total formulation; Polyurethane, 0-25% Cyanoacrylate, 0-10% Acrylates, 0-20% Vinyl acetate, 0-20% Silicone, 0-10% 2-ethylhexyl acrylate (2-EHA), 0-20% N-vinylpyrrolidone (NVP), 0-10% Acrylic acid, 0-10% Butyl acrylate, 0-20% Polyvinyl alcohol (PVA), 0-10% Polyvinylpyrrolidone (PVP), 0-10% Polyacrylamide (PAM), 0-10% Hydroxyethyl cellulose (HEC), 0-10% Carboxymethyl cellulose (CMC), 0-10% Ethylene-vinyl acetate (EVA), 0-20% Acrylonitrile-butadiene-styrene (ABS), 0-20% Styrene-acrylic (SA), 0-20% Vinyl acetate-ethylene (VAE), 0-20% Acrylic acid-ethylene (AE), 0-20%. In certain situations, the non-active ingredients can also be combined with the active ingredients during preparation of formulation.

According to another preferred embodiment, the formulation of the present invention also contains at least one the active ingredient selected from group consisting of anti-viral, amino acids and vitamin ingredients such as Docosanol, L-lysine, Acyclovir, Penciclovir, Zinc, and Magnesium. More specifically, composition comprises suitable weights of total formulation; Docosanol 0% to 10%, L-lysine 0% to 10%, Penciclovir 0% to 5%, Acyclovir 0% to 5%,
Magnesium 0% to 10% and Zinc 0% to 10%. The active ingredient can be mixed with the anti-septic compound and the non-active ingredients during preparation of formulation.

According to the present invention, the formulation of the liquid discreet patch may further include an additional antibacterial compound/ingredient selected from the group consisting of Silver, Zinc oxide, Copper oxide, Titanium dioxide, Magnesium oxide, Chitosan, Carbon, Quaternary ammonium salts, Triclosan, Chlorhexidine, Iodine complexes, Polyhexamethylene biguanide (PHMB), Benzalkonium chloride, Cetylpyridinium chloride, and Tea tree oil, not limited to nanoparticle variants. More specifically, composition comprises suitable weights of total formulation; Silver nanoparticles: 0.01%-5%, Zinc oxide nanoparticles: 0.5%-5%, Copper oxide nanoparticles: 0.01%-1%, Titanium dioxide nanoparticles: 0.5%-5%, Magnesium oxide nanoparticles: 0.01%-1%, Chitosan nanoparticles: 0.1%-1%, Carbon nanoparticles: 0.1%-1%, Quaternary ammonium salts: 0.5%-5%, Triclosan: 0.1%-1%, Chlorhexidine: 0.1%-1%, Iodine complexes: 0.1%-1%, Polyhexamethylene biguanide (PHMB): 0.01%-0.5%, Benzalkonium chloride: 0.1%-1%, Cetylpyridinium chloride: 0.1%-1%, Tea tree oil: 0.5%-2%.

According to the present invention, the liquid discreet patch is applied on to the surface of the lesion as a liquid, positioning the discreet patch on to a lesion surface and once dry, maintaining contact between the discreet dried film patch with the lesion surface for at least 1 hour, but no longer than 12 hours. Subsequent patches may be applied as needed and maintained in contact with the lesion surface, until such time as epithelialization of the lesion surface is completed.

More specifically, the liquid discreet patch is applied as a liquid over the affected dermal region, typically of the mouth and facial area. The liquid discreet patch formulation is designed to dry and form a flexible, porous, translucent film barrier that allows moisture to pass through the patch and evaporate to maintain adhesion and a dry surface area. The liquid discreet patch can also help prevent the growth and spread of the virus by acting as a barrier for external aggravations such as physical touch, bacteria, dirt, etc. reducing interaction and reducing/eliminating discomfort.

In accordance with the invention, the liquid discreet patch is characterized by consisting of a single layer with absence of any additional secondary or tertiary structures or elements such as a backing layers and wound dressings. The liquid discreet patch is configured to deliver the active ingredients at the site of the cold sore outbreaks or lesions and improves upon existing products with a pain-free water-based adhesive composition.

Accordingly, according to a preferred embodiment, the formulation of the present invention where the thickness of the liquid discreet patch is configured in between 5 and 1300 microns to maintain translucent nature and low visibility of the patch after application on to the surface of the lesion.

According to a preferred embodiment, the liquid discreet patch can be applied to a clean surface area as soon as the afflicted recognizes an oncoming outbreak and should remain in contact with the cold sore/lesion for a time up to 12 hours but no less than 2 hours. The patch can be maintained in contact with the lesion surface until such time as the outbreak has been healed and/or the associated sensations and side effects have subsided.

In accordance with the invention, the liquid discreet patch can be prepared by a two-step process at room temperature. The First step consists of the mixing of one or more the non-active ingredients while stirring at room temperature for about 30 minutes until it gets dissolves with mixing speed is 300-1000 rpm. The second step consist of the mixing of active ingredients and anti-bacterial compounds and/or non-active ingredients separately in the order deter-mined by the formulation at room temperature with a mixing speed of 300-1000 rpm. A total mixing time is 60 minutes or until all the ingredients dissolve completely to obtained a transparent solution.

### EXAMPLES

Several Examples are set forth below. The claims should not be considered to be limited to the details thereof.

### Example 1

Water-Based Liquid Patch with Docosanol 10%:

| Ingredient | Weight |
|---|---|
| Water | 82.24% |
| Glycerin | 5.00% |
| Carbomer | 1.00% |
| Triethanolamine | 1.00% |
| Docosanol | 10.00% |
| Benzalkonium chloride | 0.01% |
| Sodium hydroxide | 0.10% |
| EDTA | 0.05% |
| Methylparaben | 0.30% |
| Propylparaben | 0.20% |
| Vitamin E | 0.10% |

1. Process of preparation:
   Weigh out the appropriate amount of Water, Glycerin, Carbomer, Triethanolamine, Docosanol, Benzalkonium chloride, Sodium hydroxide, EDTA, Methylparaben, Propylparaben, and Vitamin E according to the given percentages. For example, if you want to prepare 100 grams of the liquid bandage, you would need:
      82.24 grams of Water
      5 grams of Glycerin
      1 gram of Carbomer
      1 gram of Triethanolamine
      10 grams of Docosanol
      0.01 grams of Benzalkonium chloride
      0.10 grams of Sodium hydroxide
      0.05 grams of EDTA
      0.30 grams of Methylparaben
      0.20 grams of Propylparaben
      0.10 grams of Vitamin E
2. In a clean and sanitized mixing vessel, add Water.
3. Sprinkle Carbomer over the surface of the water, allowing it to fully hydrate before proceeding. Agitation or mixing may be used to speed up the hydration process.
4. Add Glycerin to the mixing vessel and mix until fully homogenized with the Carbomer-water mixture.
5. Add Triethanolamine dropwise to the mixture while mixing continuously until a pH of 6.5-7.0 is achieved.
6. Add Docosanol to the mixture and mix until fully homogenized.
7. Add Benzalkonium chloride, Sodium hydroxide, EDTA, Methylparaben, Propylparaben, and Vitamin E to the mixture while mixing continuously until fully homogenized.
8. Continue mixing until the mixture has reached a uniform viscosity and consistency.
9. Check the pH of the mixture and adjust if necessary. The pH should be between 6.5-7.0.
10. Once the mixture has reached the desired consistency, transfer it to a suitable container for storage and use. A dropper bottle or squeeze bottle would be ideal for this application.
11. Label the container with the product name, ingredients, and any necessary usage instructions or warnings.

### Example 2

Water-Based Liquid Patch with L-Lysine 10%:

| Ingredient | Weight |
|---|---|
| Water | 80.25% |
| Glycerin | 5.00% |
| PVP | 1.00% |
| Acrylic acid | 1.00% |
| L-Lysine | 10.00% |
| Magnesium oxide | 2.00% |
| Zinc oxide | 0.10% |
| Benzalkonium chloride | 0.05% |
| Sodium hydroxide | 0.05% |
| EDTA | 0.05% |
| Methylparaben | 0.30% |
| Propylparaben | 0.20% |

### Process of Preparation:

1. Weigh out the appropriate amount of Water, Glycerin, PVP, Acrylic acid, L-Lysine, Magnesium oxide, Zinc oxide, Benzalkonium chloride, Sodium hydroxide, EDTA, Methylparaben, and Propylparaben according to the given percentages. For example, if you want to prepare 100 grams of the liquid bandage, you would need:
   80.25 grams of Water
   5 grams of Glycerin
   1 gram of PVP
   1 gram of Acrylic acid
   10 grams of L-Lysine
   2 grams of Magnesium oxide
   0.10 grams of Zinc oxide
   0.05 grams of Benzalkonium chloride
   0.05 grams of Sodium hydroxide
   0.05 grams of EDTA
   0.30 grams of Methylparaben
   0.20 grams of Propylparaben
2. Heat the water to about 80-85° C.
3. Slowly sprinkle the PVP into the heated water while continuously stirring until it dissolves completely.
4. Add the Acrylic acid to the mixture and stir until it is fully dispersed.
5. Add the L-Lysine to the mixture and stir until it is fully dissolved.
6. Add the Magnesium oxide to the mixture and stir until it is evenly dispersed.
7. Add the Zinc oxide to the mixture and stir until it is evenly dispersed.
8. Add the Benzalkonium chloride, Sodium hydroxide, EDTA, Methylparaben, and Propylparaben to the mixture, stirring well after each addition.
9. Check the pH of the mixture and adjust if necessary. The pH should be between 6.0-7.0.
10. Add the Glycerin to the mixture and stir until it is evenly distributed.
11. Continue stirring the mixture until it cools down to room temperature and becomes thick and viscous.
12. Once the mixture has reached the desired consistency, transfer it to a suitable container for storage and use. A dropper bottle or squeeze bottle would be ideal for this application.
13. Label the container with the product name, ingredients, and any necessary usage instructions or warnings.

### Example 3

Water-Based Liquid Patch with Docosanol 10% and Silver Nanoparticles:

| Ingredient | Weight |
|---|---|
| Water | 76% |
| Polyacrylate polymer | 5% |
| Glycerin | 5% |
| Carbomer | 1% |
| Triethanolamine | 1% |
| Docosanol | 10% |
| Silver nanoparticles | 1% |
| Benzalkonium chloride | 0.01% |
| Sodium hydroxide | 0.10% |
| EDTA | 0.05% |
| Methylparaben | 0.30% |
| Propylparaben | 0.20% |
| Vitamin E | 0.10% |

### Process of Preparation:

1. Weigh out the appropriate amount of Water, Polyacrylate polymer, Glycerin, Carbomer, Triethanolamine, Docosanol, Silver nanoparticles, Benzalkonium chloride, Sodium hydroxide, EDTA, Methylparaben, Propylparaben, and Vitamin E according to the given percentages. For example, if you want to prepare 100 grams of the liquid bandage, you would need:
   76 grams of Water
   5 grams of Polyacrylate polymer
   5 grams of Glycerin
   1 gram of Carbomer
   1 gram of Triethanolamine
   10 grams of Docosanol
   1 gram of Silver nanoparticles
   0.01 grams of Benzalkonium chloride
   0.10 grams of Sodium hydroxide
   0.05 grams of EDTA
   0.30 grams of Methylparaben
   0.20 grams of Propylparaben
   0.10 grams of Vitamin E
2. Heat the water to about 80-85° C.
3. Slowly sprinkle the Polyacrylate polymer into the heated water while continuously stirring until it dissolves completely.
4. Add the Glycerin to the mixture and stir well.
5. Sprinkle the Carbomer into the mixture and stir until it is fully dispersed and hydrated. This may take some time and patience, as Carbomer can be difficult to dissolve.
6. Add the Triethanolamine to the mixture and stir until the pH reaches 6.5-7.5.
7. Add the Docosanol to the mixture and stir until it is fully dissolved.
8. Add the Silver nanoparticles to the mixture and stir until they are evenly dispersed.
9. Add the Benzalkonium chloride, Sodium hydroxide, EDTA, Methylparaben, Propylparaben, and Vitamin E to the mixture, stirring well after each addition.
10. Check the pH of the mixture again and adjust if necessary.
11. Continue stirring the mixture until it cools down to room temperature and becomes thick and viscous.
12. Once the mixture has reached the desired consistency, transfer it to a suitable container for storage and use. A dropper bottle or squeeze bottle would be ideal for this application.
13. Label the container with the product name, ingredients, and any necessary usage instructions or warnings.

### Example 4

Water-Based Liquid Patch with Docosanol 10% and Silver Nanoparticles:

| Ingredient | Weight |
|---|---|
| Water | 82% |
| Docosanol | 10% |
| Silver nanoparticles | 0.05% |
| Polyacrylate polymer | 8% |

### Process of Preparation:

1. Weigh out the appropriate amount of water, Docosanol, Silver nanoparticles, and Polyacrylate polymer according to the given percentages. For example, if you want to prepare 100 grams of the liquid bandage, you would need:
   82 grams of water
   10 grams of Docosanol
   0.05 grams of Silver nanoparticles
   8 grams of Polyacrylate polymer
2. Heat the water to about 80-85° C. to dissolve the Polyacrylate polymer. Polyacrylate polymer is water-soluble, but it may take some time and heat to dissolve completely.
3. Once the Polyacrylate polymer is dissolved, reduce the heat and add the Docosanol. Continue to stir the mixture until the Docosanol is fully dissolved.
4. Add the Silver nanoparticles to the mixture and continue to stir until they are evenly dispersed.
5. Let the mixture cool to room temperature while continuing to stir. The liquid patch should start to thicken and become more viscous as it cools.
6. Once the mixture has cooled and thickened to the desired consistency, transfer it to a suitable container for storage and use. A dropper bottle or squeeze bottle would be ideal for this application.
7. Label the container with the product name, ingredients, and any necessary usage instructions or warnings.

### Example 5

Water-Based Liquid Patch with Docosanol 10% and L-Lysine:

| Ingredient | Weight |
|---|---|
| Water | 70% |
| Polyvinylpyrrolidone (PVP) | 5% |
| Polyvinyl alcohol (PVA) | 5% |
| Acrylic acid-ethylene (AE) copolymer | 10% |
| Docosanol | 5% |
| L-lysine | 5% |

### Process of Preparation:

1. Weigh out the appropriate amount of Water, Polyvinylpyrrolidone (PVP), Polyvinyl alcohol (PVA), Acrylic acid-ethylene (AE) copolymer, Docosanol, and L-lysine according to the given percentages. For example, if you want to prepare 100 grams of the liquid bandage, you would need:
   70 grams of Water
   5 grams of Polyvinylpyrrolidone (PVP)
   5 grams of Polyvinyl alcohol (PVA)
   10 grams of Acrylic acid-ethylene (AE) copolymer
   5 grams of Docosanol
   5 grams of L-lysine
2. In a clean and sanitized mixing vessel, add Water.
3. Add Polyvinylpyrrolidone (PVP) and Polyvinyl alcohol (PVA) to the mixing vessel and mix until fully dissolved.
4. Add Acrylic acid-ethylene (AE) copolymer to the mixture and mix until fully dispersed.
5. Add Docosanol and L-lysine to the mixture and mix until fully homogenized.
6. Continue mixing until the mixture has reached a uniform viscosity and consistency.
7. Once the mixture has reached the desired consistency, transfer it to a suitable container for storage and use. A dropper bottle or squeeze bottle would be ideal for this application.
8. Label the container with the product name, ingredients, and any necessary usage instructions or warnings.

### Example 6

Water-Based Liquid Patch with Penciclovir 5%, Magnesium 2%, and Zinc 1%:

| Ingredient | Weight |
|---|---|
| Water | 70% |
| Polyurethane | 5% |
| Acrylates | 5% |
| N-vinylpyrrolidone (NVP) | 5% |
| 2-ethylhexyl acrylate (2-EHA) | 5% |
| Hydroxyethyl cellulose (HEC) | 2% |
| Penciclovir | 5% |
| Magnesium | 2% |
| Zinc | 1% |

### Process of Preparation:

1. Weigh out the appropriate amount of each ingredient according to the given percentages. For example, if you want to prepare 100 grams of the liquid bandage, you would need:
   70 grams of Water
   5 grams of Polyurethane
   5 grams of Acrylates
   5 grams of N-vinylpyrrolidone (NVP)
   5 grams of 2-ethylhexyl acrylate (2-EHA)
   2 grams of Hydroxyethyl cellulose (HEC)
   5 grams of Penciclovir
   2 grams of Magnesium
   1 gram of Zinc
2. In a clean and sanitized mixing vessel, add Water.
3. Add Polyurethane, Acrylates, N-vinylpyrrolidone (NVP), 2-ethylhexyl acrylate (2-EHA), Hydroxyethyl cellulose (HEC), Zinc, and Magnesium to the mixing vessel and mix until fully dispersed.
4. Add Penciclovir to the mixture and mix until fully homogenized.
5. Continue mixing until the mixture has reached a uniform viscosity and consistency.
6. Once the mixture has reached the desired consistency, transfer it to a suitable container for storage and use. A dropper bottle or squeeze bottle would be ideal for this application.
7. Label the container with the product name, ingredients, and any necessary usage instructions or warnings.

### Example 7

Water-Based Liquid Patch with Acyclovir 5% and Zinc 5%:

| Ingredient | Weight |
|---|---|
| Water | 74% |
| Polyacrylamide (PAM) | 2% |
| Carboxymethyl cellulose (CMC) | 2% |
| Ethylene-vinyl acetate (EVA) | 10% |
| Acrylonitrile-butadiene-styrene (ABS) | 2% |
| Acyclovir | 5% |
| Zinc | 5% |

### Process of Preparation:

1. Weigh out the appropriate amount of Water, Polyacrylamide (PAM), Carboxymethyl cellulose (CMC), Ethylene-vinyl acetate (EVA), Acrylonitrile-butadiene-styrene (ABS), Acyclovir, and Zinc according to the given percentages. For example, if you want to prepare 100 grams of the formula, you would need:
   74 grams of Water
   2 grams of Polyacrylamide (PAM)
   2 grams of Carboxymethyl cellulose (CMC)
   10 grams of Ethylene-vinyl acetate (EVA)
   2 grams of Acrylonitrile-butadiene-styrene (ABS)
   5 grams of Acyclovir
   5 grams of Zinc
2. In a clean and sanitized mixing vessel, add Water.
3. Add Polyacrylamide (PAM) and Carboxymethyl cellulose (CMC) to the mixing vessel and mix until fully dissolved.
4. Add Ethylene-vinyl acetate (EVA) and Acrylonitrile-butadiene-styrene (ABS) to the mixture and mix until fully dispersed.
5. Add Acyclovir and Zinc to the mixture and mix until fully homogenized.
6. Continue mixing until the mixture has reached a uniform viscosity and consistency.
7. Once the mixture has reached the desired consistency, transfer it to a suitable container for storage and use. A dropper bottle or squeeze bottle would be ideal for this application.
8. Label the container with the product name, ingredients, and any necessary usage instructions or warnings.

### Example 8

Water-Based Liquid Patch with Docosanol 10%, Magnesium 1%, and Zinc 0.001%:

| Ingredient | Weight |
|---|---|
| Water | 72% |
| Polyvinyl acetate (PVA) | 5% |
| Styrene-acrylic (SA) copolymer | 10% |
| 2-ethylhexyl acrylate (2-EHA) | 2% |
| Docosanol | 10% |
| Magnesium | 1% |
| Zinc | 0.001% |

### Process of Preparation:

1. Weigh out the appropriate amount of Water, Polyvinyl acetate (PVA), Styrene-acrylic (SA) copolymer, 2-ethylhexyl acrylate (2-EHA), Docosanol, Magnesium, and Zinc according to the given percentages. For example, if you want to prepare 100 grams of the formulation, you would need:
   72 grams of Water
   5 grams of Polyvinyl acetate (PVA)
   10 grams of Styrene-acrylic (SA) copolymer
   2 grams of 2-ethylhexyl acrylate (2-EHA)
   10 grams of Docosanol
   1 gram of Magnesium
      1. gram of Zinc
2. In a clean and sanitized mixing vessel, add Water.
3. Add Polyvinyl acetate (PVA) to the mixing vessel and mix until fully dissolved.
4. Add Styrene-acrylic (SA) copolymer to the mixture and mix until fully dispersed.
5. Add 2-ethylhexyl acrylate (2-EHA) to the mixture and mix until fully homogenized.
6. Add Docosanol, Magnesium, and Zinc to the mixture in that order, mixing thoroughly between each addition until fully homogenized.
7. Continue mixing until the mixture has reached a uniform viscosity and consistency.
8. Once the mixture has reached the desired consistency, transfer it to a suitable container for storage and use. A dropper bottle or squeeze bottle would be ideal for this application.
9. Label the container with the product name, ingredients, and any necessary usage instructions or warnings.

### Example 9

Water-Based Liquid Patch with Penciclovir 1% and Zinc 10%:

| Ingredient | Weight |
|---|---|
| Water | 74% |
| Polyurethane | 5% |
| Acrylates | 5% |
| N-vinylpyrrolidone (NVP) | 5% |
| 2-ethylhexyl acrylate (2-EHA) | 5% |
| Penciclovir | 1% |
| Zinc | 5% |

### Process of Preparation:

1. Weigh out the appropriate amount of Water, Polyurethane, Acrylates, N-vinylpyrrolidone (NVP), 2-ethylhexyl acrylate (2-EHA), Penciclovir, and Zinc according to the given percentages. For example, if you want to prepare 100 grams of the formula, you would need:
   74 grams of Water
   5 grams of Polyurethane
   5 grams of Acrylates
   5 grams of N-vinylpyrrolidone (NVP)
   5 grams of 2-ethylhexyl acrylate (2-EHA)
   1 gram of Penciclovir
   5 grams of Zinc
2. In a clean and sanitized mixing vessel, add Water.
3. Add Polyurethane to the mixing vessel and mix until fully dispersed.
4. Add Acrylates to the mixture and mix until fully dissolved.
5. Add N-vinylpyrrolidone (NVP) to the mixture and mix until fully dissolved.
6. Add 2-ethylhexyl acrylate (2-EHA) to the mixture and mix until fully dissolved.
7. Add Penciclovir to the mixture and mix until fully dispersed.
8. Add Zinc to the mixture and mix until fully dispersed.
9. Continue mixing until the mixture has reached a uniform viscosity and consistency.
10. Once the mixture has reached the desired consistency, transfer it to a suitable container for storage and use. A dropper bottle or squeeze bottle would be ideal for this application.
11. Label the container with the product name, ingredients, and any necessary usage instructions or warnings.

### Example 10

Water-Based Liquid Patch with Acyclovir 1% to 5%:

| Ingredient | Weight |
|---|---|
| Water | 75% |
| Polyvinylpyrrolidone (PVP) | 5% |
| Polyvinyl alcohol (PVA) | 5% |
| Acrylic acid-ethylene (AE) copolymer | 10% |
| Acyclovir | 5% |

### Process of Preparation:

1. Weigh out the appropriate amount of Water, Polyvinylpyrrolidone (PVP), Polyvinyl alcohol (PVA), Acrylic acid-ethylene (AE) copolymer, and Acyclovir according to the given percentages. For example, if you want to prepare 100 grams of the liquid bandage, you would need:
   75 grams of Water
   5 grams of Polyvinylpyrrolidone (PVP)
   5 grams of Polyvinyl alcohol (PVA)
   10 grams of Acrylic acid-ethylene (AE) copolymer
   5 grams of Acyclovir
2. In a clean and sanitized mixing vessel, add Water.
3. Gradually sprinkle Polyvinylpyrrolidone (PVP) and Polyvinyl alcohol (PVA) into the mixing vessel while stirring continuously. This helps to prevent clumping and ensures even distribution of the polymers in the water.
4. Add Acrylic acid-ethylene (AE) copolymer to the mixture and stir until it is fully dispersed. This may take several minutes of stirring.
5. Add Acyclovir to the mixture and stir until it is fully dissolved.
6. Heat the mixture to 80-85° C. while stirring continuously until it forms a clear solution. This temperature range helps to dissolve any remaining solids and improves the viscosity of the solution.
7. Allow the mixture to cool to room temperature while stirring occasionally to prevent sedimentation.
8. Once the mixture has cooled, transfer it to a suitable container for storage and use. A dropper bottle or squeeze bottle would be ideal for this application.
9. Label the container with the product name, ingredients, and any necessary usage instructions or warnings.

### Example 11

Water-Based Liquid Patch with Penciclovir 1% to 5%:

| Ingredient | Weight |
|---|---|
| Water | 75% |
| Polyurethane | 5% |
| Acrylates | 5% |
| N-vinylpyrrolidone (NVP) | 5% |
| 2-ethylhexyl acrylate (2-EHA) | 5% |
| Penciclovir | 5% |

### Process of Preparation:

1. Weigh out the appropriate amount of Water, Polyurethane, Acrylates, N-vinylpyrrolidone (NVP), 2-ethylhexyl acrylate (2-EHA), and Penciclovir according to the given percentages. For example, if you want to prepare 100 grams of the formulation, you would need:
   75 grams of Water
   5 grams of Polyurethane
   5 grams of Acrylates
   5 grams of N-vinylpyrrolidone (NVP)
   5 grams of 2-ethylhexyl acrylate (2-EHA)
   5 grams of Penciclovir
2. In a clean and sanitized mixing vessel, add Water.
3. Add Polyurethane to the mixing vessel and mix until fully dissolved.
4. Add Acrylates and mix until fully dispersed.
5. Add N-vinylpyrrolidone (NVP), 2-ethylhexyl acrylate (2-EHA), and Penciclovir to the mixture and mix until fully homogenized.
6. Heat the mixture to a suitable temperature to activate the polymerization process, if required by the specific type of Polyurethane being used. If no activation is needed, skip this step.
7. Continue mixing until the mixture has reached a uniform viscosity and consistency.
8. Once the mixture has reached the desired consistency, transfer it to a suitable container for storage and use. A dropper bottle or squeeze bottle would be ideal for this application.
9. Label the container with the product name, ingredients, and any necessary usage instructions or warnings.

### Example 12

Water-Based Liquid Patch with L-Lysine 5% to 10%:

| Ingredient | weight |
|---|---|
| Water | 75% |
| Polyacrylamide (PAM) | 2% |
| Carboxymethyl cellulose (CMC) | 2% |
| Ethylene-vinyl acetate (EVA) | 10% |
| Acrylonitrile-butadiene-styrene (ABS) | 2% |
| L-lysine | 9% |

### Process of Preparation:

1. Weigh out the appropriate amount of Water, Polyacrylamide (PAM), Carboxymethyl cellulose (CMC), Ethylene-vinyl acetate (EVA), Acrylonitrile-butadiene-styrene (ABS), and L-lysine according to the given percentages. For example, if you want to prepare 100 grams of the formula, you would need:
   75 grams of Water
   2 grams of Polyacrylamide (PAM)
   2 grams of Carboxymethyl cellulose (CMC)
   10 grams of Ethylene-vinyl acetate (EVA)
   2 grams of Acrylonitrile-butadiene-styrene (ABS)
   9 grams of L-lysine
2. In a clean and sanitized mixing vessel, add Water.
3. Add Polyacrylamide (PAM) and Carboxymethyl cellulose (CMC) to the mixing vessel and mix until fully dissolved.
4. Add Ethylene-vinyl acetate (EVA) and Acrylonitrile-butadiene-styrene (ABS) to the mixture and mix until fully dispersed.
5. Add L-lysine to the mixture and mix until fully homogenized.
6. Continue mixing until the mixture has reached a uniform viscosity and consistency.
7. Once the mixture has reached the desired consistency, transfer it to a suitable container for storage and use. A bottle or container with a tight-fitting lid would be ideal for this application.
8. Label the container with the formula name, ingredients, and any necessary usage instructions or warnings.

1. A formulation of a liquid discrete patch for the treatment of cold sores comprising;
a. a translucent liquid adhesive, characterized in that the alcohol content of less than 25% by weight,
b. least one active ingredient,
c. a non-active ingredient, and
d. an anti-bacterial/microbial compound
Whereas, said translucent liquid adhesive is mixed with said active ingredient and configured to form a layer without any additional backing layer.

2. The formulation of a liquid discrete patch according to the claim 1, wherein the translucent liquid adhesive is composed of a water-base which includes the non-active ingredient(s).

3. The formulation of a liquid discrete patch according to the claim 1, wherein the non-active ingredient is selected from the group consisting of Polyurethane, cyanoacrylate, acrylates, vinyl acetate, silicone, 2-ethylhexyl acrylate (2-EHA), N-vinylpyrrolidone (NVP), acrylic acid, vinyl acetate, butyl acrylate, polyvinyl alcohol (PVA), polyvinylpyrrolidone (PVP), polyacrylamide (PAM), hydroxyethyl cellulose (HEC), carboxymethyl cellulose (CMC), ethylene-vinyl acetate (EVA), acrylonitrile-butadiene-styrene (ABS), styrene-acrylic (SA), vinyl acetate-ethylene (VAE), acrylic acid-ethylene (AE).

4. The formulation of a liquid discrete patch according to the claim 1, wherein the active ingredient is selected from the group consisting of Docosanol, L-lysine, Acyclovir, Penciclovir, Zinc and Magnesium.

5. The formulation of a liquid discrete patch according to the claim 1, wherein the anti-bacterial/anti-microbial compound is selected from the group consisting of silver nanoparticles, Zinc oxide nanoparticles, Copper oxide nanoparticles, Titanium dioxide nanoparticles, Magnesium oxide nanoparticles, Chitosan nanoparticles, Carbon nanoparticles, Quaternary ammonium salts, Triclosan, Chlorhexidine, Iodine complexes, Polyhexamethylene biguanide (PHMB), Benzalkonium chloride, Cetylpyridinium chloride, Tea tree oil.

6. The formulation of a liquid discrete patch according to the claim 1, wherein the ingredients are water 80% by weight, glycerin 5% by weight, carbomer 1% by weight, Triethanolamine 1% by weight, Docosanol 10% by weight, Benzalkonium chloride 0.01% by weight, Sodium hydroxide 0.1% by weight, EDTA 0.05% by weight, Methylparaben 0.3% by weight, Propylparaben 0.2% by weight, Vitamin E 0.1% by weight.

7. The formulation of a liquid discrete patch according to the claim 1, wherein the ingredients are Water 85% by weight, Glycerin 5% by weight, PVP 1% by weight, acrylic acid 1% by weight, L-lysine 10% by weight, Magnesium oxide 2% by weight, Zinc oxide 0.1% by weight, Benzalkonium chloride 0.05% by weight, Sodium hydroxide 0.05% by weight, EDTA 0.05% by weight, Methylparaben 0.3% by weight, Propylparaben 0.2% by weight.

8. The formulation of a liquid discrete patch according to the claim 1, wherein the ingredients are Water 70% by weight, Polyacrylate polymer 5% by weight, Glycerin 5% by weight, Carbomer 1% by weight, Triethanolamine 1% by weight, Docosanol 10% by weight, silver nanoparticles 1% by weight, Benzalkonium chloride 0.01% by weight, Sodium hydroxide 0.1% by weight, EDTA 0.05% by weight, Methylparaben 0.3% by weight, Propylparaben 0.2% by weight, Vitamin E 0.1% by weight.

9. The formulation of a liquid discrete patch according to the claim 1, wherein the ingredients are Water 79% by weight, 85% by weight, Docosanol 10% by weight, silver nanoparticles 0.05% by weight, Polyacrylate polymer 4% by weight, 10% by weight.

10. The formulation of a liquid discrete patch according to the claim 1, wherein the ingredients are Water 75% by weight, Polyvinylpyrrolidone (PVP) 5% by weight, Polyvinyl alcohol (PVA) 5% by weight, acrylic acid-ethylene (AE) copolymer 10% by weight, Docosanol 10% by weight, L-lysine 5% by weight.

11. The formulation of a liquid discrete patch according to the claim 1, wherein the ingredients are Water 75% by weight, Polyurethane 5% by weight, Acrylates 5% by weight, N-vinylpyrrolidone (NVP) 5% by weight, 2-ethylhexyl acrylate (2-EHA) 5% by weight, Hydroxyethyl cellulose (HEC) 2% by weight, Penciclovir 5% by weight, Magnesium 2% by weight, Zinc 1% by weight.

12. The formulation of a liquid discrete patch according to the claim 1, wherein the ingredients are Water 80% by weight, Polyacrylamide (PAM) 2% by weight, Carboxymethyl cellulose (CMC) 2% by weight, Ethylene-vinyl acetate (EVA) 10% by weight, Acrylonitrile-butadiene-styrene (ABS) 2% by weight, Acyclovir 5% by weight, Zinc 5% by weight.

13. The formulation of a liquid discrete patch according to the claim 1, wherein the ingredients are Water 80% by weight, Polyvinyl acetate (PVA) 5% by weight, Styrene-acrylic (SA) copolymer 10% by weight, 2-ethylhexyl acrylate (2-EHA) 2% by weight, Docosanol 10% by weight, Magnesium 1% by weight, Zinc 0.001% by weight.

14. The formulation of a liquid discrete patch according to the claim 1, wherein the ingredients are Water 80% by weight, Polyurethane 5% by weight, Acrylates 5% by weight, N-vinylpyrrolidone (NVP) 5% by weight, 2-ethylhexyl acrylate (2-EHA) 5% by weight, Penciclovir 1% by weight, Zinc 10% by weight.

15. The formulation of a liquid discrete patch according to the claim 1, wherein the ingredients are Water 75% by weight, Polyvinylpyrrolidone (PVP) 5% by weight, Polyvinyl alcohol (PVA) 5% by weight, Acrylic acid-ethylene (AE) copolymer 10% by weight, Acyclovir 1% by weight, to 5% by weight.

16. The formulation of a liquid discrete patch according to the claim 1, wherein the ingredients are Water 75% by weight, Polyurethane 5% by weight, Acrylates 5% by weight, N-vinylpyrrolidone (NVP) 5% by weight, 2-ethylhexyl acrylate (2-EHA) 5% by weight, Penciclovir 1% by weight, to 5% by weight.

17. The formulation of a liquid discrete patch according to the claim 1, wherein the ingredients are Water 75% by weight, Polyacrylamide (PAM) 2% by weight, Carboxymethyl cellulose (CMC) 2% by weight, Ethylene-vinyl acetate (EVA) 10% by weight, Acrylonitrile-butadiene-styrene (ABS) 2% by weight, L-lysine 5% by weight, to 10% by weight.

17. A method of use of a liquid discrete patch of claim 1, the method comprising:
(a) positioning the liquid discrete patch on to a lesion surface and maintaining contact between the liquid discrete patch with the lesion surface for at least 1 hours, but no longer than 12 hours.
(b) delivering subsequent patches and maintaining said subsequent patches in contact with the lesion surface, until such time as epithelialization of the lesion surface is completed.

18. The formulation of a liquid discrete patch according to the claim 1, wherein the liquid discrete patch characterized by being applied as a liquid and once dried, forming a translucent film barrier with a thickness of between 5 and 1300 microns.

19. The formulation of a liquid discrete patch according to the claim 1, wherein liquid discrete patch characterized as being flexible and porous with the ability to allow moisture to pass through and evaporate, maintaining a dry surface that is unaffected by movement in regular wear.
